(19) United States
(12) Patent Application Publication
(10) Pub. No.: US 2010/0221326 A1
(43) Pub. Date: Sep. 2, 2010

(54) PHARMACEUTICAL COMPOSITIONS FOR ORAL USE FOR TREATING PATIENTS AFFECTED BY OBESITY

(75) Inventors: Guido Vandoni, Milano (IT); Heny Cassia Zaniboni, Milano (IT)

Correspondence Address: KNOBBE MARTENS OLSON & BEAR LLP, 2040 MAIN STREET, FOURTEENTH FLOOR, IRVINE, CA, 92614, US

(73) Assignee: MADER S.R.L., MILANO (IT)

(21) Appl. No.: 12/678,066

(22) PCT Filed: Sep. 12, 2008
(86) PCT No.: PCT/EP08/62165

§ 371 Date: Mar. 12, 2010

(30) Foreign Application Priority Data
Sep. 12, 2007 (EP) 07425563.9

(51) Int. Cl. A61K 31/337; A61P 3/04; A61K 9/48

(52) U.S. Cl. 424/456; 514/449; 424/451

(57) ABSTRACT

Pharmaceutical compositions are for administered orally for treating patients affected by obesity. The compositions include orlistat (tetrahydrolipstatin) solubilized in pharmaceutically acceptable saturated hydrocarbons obtained from petroleum and one or more surfactants.

### STATE OF THE ART

Orlistat is an active principle belonging to the therapeutic anti-obesity agents group of formula

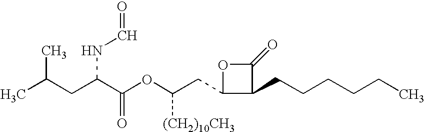

Orlistat is administered in conjunction with low calorie diets for treating patients affected by obesity having a body mass index (BMI) greater than or equal to 30 kg/m², or overweight patients with a BMI ≧28 kg/m² with associated risk factors. Orlistat is a substance which, from the technological viewpoint, has some stability problems; in this respect the active principle Orlistat is low melting (around 44° C.) and undergoes rapid hydrolysis in the presence of water or moisture. Stability studies of the raw material, under ICH conditions (25° C., 65% RH) show a 15% degradation of the active principle after 7 days' storage.

Furthermore, degradation is directly proportional to temperature; at a temperature of 40° C. and RH of 75% degradation is greater than 40%.

In order to overcome said degradation, formulations in soft capsules have been produced as described in U.S. Pat. No. 4,598,089 filed on 18 Jun. 1984 in which the active principle was dissolved in the triglyceride mixture NEOBEE M-5, then distributed into soft gelatin capsules.

Subsequently, in U.S. Pat. No. 6,004,996 filed on 6 Jan. 1998, a formulation in granules/pellets was described containing as excipient a stabilizer able to control the degree of humidity and not to degrade the Orlistat. This formulation contains excipients such as: diluents, surfactants and disintegrants.

In U.S. Pat. No. 6,358,522 filed on 10 Aug. 1999, formulations in the form of a powder and chewable tablets containing Orlistat combined with a thickening agent and an emulsifier are described.

### SUMMARY OF THE INVENTION

The present invention relates to a pharmaceutical formulation based on Orlistat solubilized in mixtures of saturated hydrocarbons obtained from petroleum. These substances due to their lipophilicity do not contain water, the Orlistat being easily solubilized therein. An ionic, non-ionic or amphoteric surfactant is then added to the Orlistat solution obtained.

Surprisingly, the formulation thus attained is found to be stable with an excellent in-vitro dissolution profile.

### DETAILED DESCRIPTION OF THE INVENTION

The invention relates to highly stable pharmaceutical preparations for treating obesity which contain Orlistat as the active principle.

The Orlistat is solubilized in mixtures of pharmaceutically acceptable saturated hydrocarbons obtained from petroleum, with an active principle:solvent ratio of between 1:0.5 and 1:10, preferably between 1:1 and 1:2.

Suitable mineral oils are paraffin oil, light paraffin oil, Liquid Paraffin and Paraffin Light Liquid (as described in the European Pharmacopeia). The Paraffin Hard product described in the European Pharmacopeia while having a waxy consistency has also shown an excellent capacity for solubilizing Orlistat, either alone or mixed with other paraffinic oils.

The mineral oil Paraffin Liquid has a relative density of 0.82-0.89.

The mineral oil Paraffin Light Liquid has a relative density of 0.810-0.875.

The product Paraffin Hard has a melting point of 50-61° C.

The Orlistat solution in saturated hydrocarbon mixtures obtained from petroleum is further supplemented with one or more ionic, non-ionic or amphoteric surfactants in a ratio of active principle:surfactant of between 1:0.001 and 1:1, preferably between 1:0.01 and 1:0.05.

Examples of suitable surfactants are sodium lauryl sulfate, Tween 20, 60, 80, alkylamidobetaine and phospholipids, either taken singly or mixed together. Other surfactants useful for implementing the present invention are: Macrogolglycerol ricinoleate, Macrogolglycerol hydroxystearate, Polysorbate 20, Polysorbate 21, Polysorbate 40, Polysorbate 61, Polysorbate 65, Polysorbate 81, Polysorbate 85, lauryl alcohol polyethanoleate, soya, maize or egg lecithins, lecithins purified to the extent of 60, 70, 80, 90 and 95% phosphatidylcholine and 80, 90 and 100% hydrogenated phosphatidylcholine.

The solution of active principle in the paraffinic oil containing the surfactant can be inserted into soft capsules or hard gelatin capsules.

Some purely illustrative examples of the invention are given below with relative solubility and stability data of the prepared products.

### EXAMPLES

### Example 1

### Soft Capsules

| Active principle: | | |
|---|---|---|
| Orlistat | 120,000 | mg |
| Excipients for the fill: | | |
| light paraffin oil | 280,140 | mg |
| Sodium lauryl sulfate | 2,805 | mg |
| Excipients for the shell: | | |
| Gelatin | 132,800 | mg |
| Glycerol | 77,200 | mg |
| Titanium Dioxide | 6,200 | mg |

Capsule characteristics: oval D

### Preparation Method

### Preparation of the Fill

0.350 kg of light paraffin oil were introduced into a stainless steel container and, stirring gently with rotating paddles, 0.0035 kg of sodium lauryl sulfate were added. Stirring was continued until complete dissolution of the surfactant.

The Orlistat (0.15 kg) was added to the solution thus obtained, gently stirring until complete dissolution.

The solution thus obtained was deaerated and maintained under vacuum.

### Preparation of the Shell

9.41 kg of purified water was introduced into a container equipped with heated jacket and stirrer, the temperature being controlled at 80° C.

While stirring vigorously the glycerol (6.76 kg) and the gelatin (12.65 kg) were added, to the warmed water until complete dissolution.

The gelatin/glycerol/water solution thus obtained was maintained under vacuum, controlling the temperature at 80° C.

The colouring agent was then added to this solution, prepared by dispersing titanium dioxide (0.59 kg) in glycerol (0.59 kg) with a turboemulsifier.

The titanium dioxide in glycerol dispersion was added to the gelatin/glycerol/water solution to disperse the colouring while maintaining the temperature always at 80° C.

### Encapsulation

By means of an encapsulating machine, the coating mass was placed in the two appropriate hoppers and passed through laminators at 60° C. so as to form the coating.

The sheets were moulded through suitable rollers and 403.0 mg of the Orlistat solution were inserted between the two gelatin sheets with a suitable dosing needle.

### Drying

The thus prepared capsules were placed in rotating drying tubes and, after pre-drying, were positioned on suitable racks in temperature controlled cupboards at 21-22° C. and 20% RH for 1-2 days.

### Solubility Tests

The dissolution profile was characterized in-vitro in accordance with the following methodology:
Method: USP
Apparatus: Paddle II
Medium: phosphate buffer pH 6.9
Volume: 900 ml
Rotation speed: 100 r.p.m
Temperature: 37° C.
   Solubility results (%)

| Sample | After 10′ | After 20′ | After 30′ | After 45′ |
|---|---|---|---|---|
| No. 1 | 12.7 | 55.6 | 75.9 | 85.8 |
| No. 2 | 15.2 | 50.7 | 79.9 | 88.7 |
| No. 3 | 11.0 | 60.4 | 80.1 | 86.7 |
| No. 4 | 16.4 | 58.8 | 77.9 | 86.4 |
| No. 5 | 14.4 | 61.1 | 76.1 | 89.3 |
| No. 6 | 14.9 | 60.2 | 80.7 | 95.1 |

The Orlistat-based preparations in soft capsules were subjected to ICH stability studies at conditions of 25° C. 60% RH, 30° C. 70% RH and 40° C. 75% RH and demonstrated excellent active principle stability.

The active principle titres were determined by analysis with HPLC Diode Array MD1510, using 70:30 methanol:water as the mobile phase, with Lichrosphere RP8 column 125×4 mm, 5 mcm (Merck), at a wavelength of 210 nm.

### Stability Results:

| | isothermal 25° C. 60% RH | | | | | |
|---|---|---|---|---|---|---|
| | T zero | 1 month | 3 months | 6 months | 9 months | 12 months |
| orlistat HPLC | 100.00% | 100.01% | 100.00% | 100.20% | 100.01% | 100.2% |
| titre | | | | | | |
| | isothermal 30° C. 70% RH | | | | | |
| | T zero | 1 month | 3 months | 6 months | 9 months | 12 months |
| orlistat HPLC | 100.00% | 100.01% | 100.00% | 100.20% | 100.01% | 100.2% |
| titre | | | | | | |

| | isothermal 40° C. 75% RH | | | |
|---|---|---|---|---|
| | T zero | 1 month | 3 months | 6 months |
| orlistat HPLC | 100.00% | 99.90% | 99.80% | 99.90% |
| titre | | | | |

### Example 2

### Soft Capsules

| | |
|---|---|
| Active principle: | |
| Orlistat | 120,000 mg |
| Excipients for the fill: | |
| paraffin oil | 263,000 mg |
| Tween 80 | 20,000 mg |
| Excipients for the capsule: | |
| Gelatin | 132,800 mg |
| Glycerol | 77,200 mg |
| Iron oxide | 6,200 mg |

Capsule characteristics: 10 oval D

### Preparation Method

### Preparation of the Fill

The paraffin oil (0.263 kg) was weighed and inserted into a stainless steel container and, stirring gently with rotating paddles, Tween 80 (0.020 kg) was added and dissolved. Stirring was continued until complete dissolution of the surfactant.

The Orlistat (0.12 kg) was added to the solution thus obtained and dissolved while gently stirring.

The solution thus obtained was deaerated and maintained under vacuum.

Preparation of the Capsule 59.41 kg of purified water were introduced into a container equipped with heated jacket and stirrer, the temperature being controlled at 80° C.

While stirring vigorously the glycerol (6.76 kg) then the gelatin (12.65 kg) were added to the thus heated water and dissolved.

The gelatin/glycerol/water solution thus obtained was maintained under vacuum and temperature controlled at 80° C.

The colouring agent, prepared by dispersing the iron oxide (0.59 kg) in glycerol (0.59 kg) with a turboemulsifier, was added to this solution.

The iron oxide in glycerol dispersion was added to the gelatin/glycerol/water solution to disperse the colouring while maintaining the temperature at 80° C.

### Encapsulation

By means of an encapsulating machine, the coating mass was placed in the two appropriate hoppers and passed through laminators at 60° C. so as to form the coating.

The sheets were passed through suitable rollers to allow moulding and 403.0 mg of the Orlistat solution were inserted between the two gelatin sheets with a suitable dosing needle.

### Drying

The already prepared capsules were placed in rotating drying tubes and, after pre-drying, were placed onto suitable racks in temperature controlled cupboards at 21-22° C. and 20% RH for 1-2 days.

### Example 3

### Hard Gelatin Capsules

| | |
|---|---|
| Active principle: | |
| Orlistat | 120,000 mg |
| Excipients: | |
| Paraffin Liquid | 100,000 mg |
| Paraffin Hard | 60,000 mg |
| Phosphatidylcholine (80) | 20,000 mg |

Capsule: size 00 white opaque, average weight 118 mg ± 7.0 mg.

### Preparation Method

### Preparation

The Paraffin Liquid (10.000 kg) and Paraffin Hard (6.000 kg) were weighed, placed in a stainless steel container and melted at a temperature of 70° C., stirring gently with rotating paddles; the phosphatidylcholine (2.000 kg) was then added and melted. Stirring was maintained until this latter dissolved completely, controlling the temperature at 30° C.

The Orlistat (12.00 kg) was added to the solution thus obtained and dissolved while gently stirring.

### Encapsulation

Using a hard capsule filling machine equipped to dispense liquids, the liquid was dispensed into hard gelatin capsules, size 00, to a weight of 300 mg/cps of fill.

1. A composition for oral use for treating patients affected by obesity comprising Orlistat (tetrahydrolipstatin) solubilized in a mixture of pharmaceutically acceptable saturated hydrocarbons obtained from petroleum, and one or more surfactants.

2. A composition as claimed in claim 1, wherein the saturated hydrocarbon mixture is paraffin oil or light paraffin oil.

3. A composition as claimed in claim 1, wherein the saturated hydrocarbon mixture is Paraffin Light Liquid, having a relative density from 0.810 to 0.875.

4. A composition as claimed in claim 1, wherein the saturated hydrocarbon mixture is Paraffin Liquid, having a relative density from 0.82 to 0.89.

5. A composition as claimed in claim 1, wherein the saturated hydrocarbon mixture is Paraffin Hard, having a melting point from 50 to 61° C.

6. A composition as claimed in claim 1, wherein the saturated hydrocarbon mixture is a varying proportion mixture of 2 or 3 products selected from the group consisting of paraffin oil, light paraffin oil, Paraffin Light Liquid, Paraffin Liquid and Paraffin Hard in all possible quantitative combinations.

7. A composition as claimed in claim 1, wherein each of the one or more surfactants is ionic, non-ionic or amphoteric.

8. A composition as claimed in claim 7, wherein the one or more surfactants is sodium lauryl sulfate alone or mixed with other surfactants.

9. A composition as claimed in claim 7, wherein the one or more surfactants are selected from the group consisting of Tween 20, Tween 60, Tween 80 and a mixture thereof.

10. A composition as claimed in claim 7, wherein the one or more surfactants is are selected from the group consisting of alkylamidobetaines and a mixture thereof.

11. A composition as claimed in claim 7, wherein the one or more surfactants are selected from the group consisting of Macrogolglycerol ricinoleate, Macrogolglycerol hydroxystearate, Polysorbate 20, Polysorbate 21, Polysorbate 40, Polysorbate 61, Polysorbate 65, Polysorbate 81, Polysorbate 85, lauryl alcohol polyethanoleate, soya, maize or egg lecithins, lecithins purified to the extent of 60%, lecithins purified to the extent of 70%, lecithins purified to the extent of 80%, lecithins purified to the extent of 90%, lecithins purified to the extent of 95%, pphosphatidylcholine, 80% hydrogenated phosphatidylcholine, 90% hydrogenated phosphatidylcholine and 100% hydrogenated phosphatidylcholine.

12. A composition as claimed in claim 1 enclosed in a soft capsule.

13. A composition as claimed in claim 1 enclosed in a hard gelatin capsule.

14. A composition as claimed in claim 1, wherein the ratio of Orlistat to the saturated hydrocarbon mixture, which acts as solvent, is between 1:0.5 and 1:10.

15. A composition as claimed in claim 13, wherein the ratio of Orlistat to the saturated hydrocarbon mixture, which acts as solvent, is between 1:1 and 1:2.

16. A composition as claimed in claim 1, wherein the ratio of Orlistat solubilized in said mixture of pharmaceutically acceptable saturated hydrocarbons obtained from petroleum:surfactant is between 1:0.001 and 1:1.

17. A composition as claimed in claim 15, wherein the ratio of Orlistat solubilized in said mixture of pharmaceutically acceptable saturated hydrocarbons obtained from petroleum:surfactant is between 1:0.01 and 1:0.05.

18. A method of treating obesity in an obese subject comprising the step of administering to said subject a composition according to claim 1.

19. A method of treating obesity in an obese subject comprising the step of administering to said subject a composition according to claim 2.

20. A method of treating obesity in an obese subject comprising the step of administering to said subject a composition according to claim 3.
